(19) United States
(12) Patent Application Publication
(10) Pub. No.: US 2016/0030705 A1
(43) Pub. Date: Feb. 4, 2016

(54) INSERTION ELEMENT FOR MEDICAL IMPLANT INSERTION DEVICE

(71) Applicant: BIOTRONIK AG, Buelach (CH)

(72) Inventor: Amir Fargahi, Buelach (CH)

(21) Appl. No.: 14/741,642

(22) Filed: Jun. 17, 2015

Related U.S. Application Data
(60) Provisional application No. 62/030,624, filed on Jul. 30, 2014.

(51) Int. Cl. A61M 25/00; A61B 17/34

(52) U.S. Cl. CPC A61M 25/005; A61M 25/0054; A61B 17/3468

(57) ABSTRACT

An insertion element configured for cooperation with an insertion device for the insertion of a medical implant into a human or animal body. The insertion element has a sleeve with a longitudinal axis. A frame has transverse elements, at least in regions, that are arranged transversely to the longitudinal axis. The transverse elements follow one another along the longitudinal axis and are integrated into the sleeve. The transverse elements are connected along the longitudinal axis to a longitudinal element. The frame (110) has a higher hardness than the sleeve.

### PRIORITY CLAIM

This application claims priority under 35 U.S.C. §119 from prior U.S. Provisional Application No. 62/030,624, filed Jul. 30, 2014.

### FIELD OF THE INVENTION

A field of the invention is insertion devices, in particular catheters, for insertion of a medical implant into a human and/or animal body.

### BACKGROUND

Medical implants are often introduced into a human and/or animal body for treatment. Permanent and long-term implants carry out replacement functions. Example implants include heart pacemakers, brain pacemakers for Parkinson's patients, cardiac implants, cochlear implants, retinal implants, dental implants, implants for joint replacement, vessel prostheses, or stents.

Before insertion into the body, implants are connected to catheters, with the aid of which the implants can be placed precisely at the site of intended use and can be released in a defined manner. For introduction into the human and/or animal body, a tube-like insertion element is used for this purpose, through which the implant is inserted by use of the insertion device. In order to reduce the loading of the vessels when the implant is introduced, an insertion element is known from US 2010/0094392 A1 that is only expanded to the necessary larger diameter when the implant passes through. The insertion element consists of two to three coaxially arranged layers, wherein the outer layer is provided with longitudinal slits, which enables the enlargement of the diameter.

EP 2 676 641 A2 discloses a reversibly expandable insertion element with a sleeve, which has regions of different resilience at the periphery. A catheter can be introduced into the body through the expandable insertion element, wherein the insertion element expands as the implant fastened to the catheter passes through and contracts again to its original smaller diameter once the implant has passed through.

### SUMMARY OF THE INVENTION

A preferred insertion element is configured to cooperate with an insertion device for the insertion of a medical implant into a human and/or animal body. The insertion element has a sleeve having a longitudinal axis. A frame has, at least in regions, transverse elements arranged transversely to the longitudinal axis. The transverse elements follow one another along the longitudinal axis and are integrated into the sleeve. The transverse elements are connected along the longitudinal axis to a longitudinal element. The frame has a higher hardness than the sleeve. In preferred embodiments, the longitudinal element is resilient and bendable. In preferred embodiments, at least one of the transverse elements is a ring element. In preferred embodiments, the sleeve contains an inner and outer part with the frame arranged therebetween.

### BRIEF DESCRIPTION OF THE DRAWINGS

The invention is explained hereinafter by way of example on the basis of exemplary embodiments illustrated in drawings, in which:

FIG. 1 schematically shows a view of an insertion device in accordance with an exemplary embodiment of the invention with an insertion element with a frame, through which an implant is channeled into a human and/or animal body;

FIG. 2 schematically shows a plan view of a frame with straight elements, which are connected to a longitudinal element; and

FIG. 3 schematically shows a curved frame with ring elements and a longitudinal element connecting the ring elements.

FIG. 4 schematically shows, in side view, a frame with ring elements that are connected by means of a longitudinal element;

FIG. 5 schematically shows, in side view, an insertion element with sleeve and indicated frame in the sleeve;

FIG. 6 schematically shows a cross section through the insertion element from FIG. 5;

FIG. 7 schematically shows, in plan view, a detail of a frame in accordance with an embodiment of the invention, in which the expandable ring elements are arranged concentrically with a gap in the periphery, such that the gaps in the periphery are covered;

FIG. 8 schematically shows, in plan view, a detail of a frame in accordance with an embodiment of the invention, with an expandable ring element with overlapping ends, such that a gap in the periphery is covered;

### DETAILED DESCRIPTION OF THE PREFERRED EMBODIMENTS

Preferred embodiment insertion elements provide improved mechanical properties and are flexible, at least in a reversible manner. A preferred insertion element is configured to cooperate with an insertion device for the insertion of a medical implant into a human and/or animal body and that has a sleeve having a longitudinal axis. A frame has, at least in regions, elements arranged transversely to the longitudinal axis. The frame elements follow one another along the longitudinal axis and are integrated into the sleeve. The frame elements are connected along the longitudinal axis to a longitudinal element. The frame has a higher hardness than the sleeve.

Such an insertion element can advantageously be used as a port for a catheter with an implant, via which the catheter and the implant can be introduced into the body. The insertion element has a high stability along its longitudinal axis due to the stable frame, such that on the one hand an advantageous flexibility is provided and on the other hand a favorable behavior under the action of a compressive force is provided when the catheter is inserted. The desired properties can be optimized by the selection of favorable material combinations and dimensions thereof.

The longitudinal element supports the insertion element in the direction of the longitudinal axis, such that the insertion element is stabilized in the event of pushing movements or pulling movements of the catheter. The longitudinal element can, for example, be welded to the elements, preferably by laser welding. The elements may be ring elements, which can preferably be expanded. The insertion element can then additionally be expanded also radially. However, the elements may also be straight elements in other embodiments. In this case, it may be advantageous when the sleeve of the insertion element has a varying resilience along the periphery, for example in the manner of an insertion element as described in EP 2 676 641 A2.

In accordance with a preferred embodiment, the longitudinal element can be bent resiliently. The longitudinal element can stabilize the curved sleeve because of its restoring force and its stability in the longitudinal direction.

In accordance with a preferred embodiment, at least one of the elements can be formed as a ring element, which can be resiliently expanded in the radial direction. An insertion element that can be expanded in a reversible manner is advantageously provided. Since the insertion element reduces its diameter again once the implant channeled into the human and/or animal body through the insertion element has passed through, the vessel loading of the body is reduced.

In accordance with a preferred embodiment, at least one of the ring elements can be curved along a closed or almost closed periphery, with the two ends are separated from one another. Ring elements of this type are easy to produce and the dimensions thereof are easily adjusted.

In accordance with a preferred embodiment, at least one ring element may have two ends overlapping at its periphery. An opening in the periphery is thus covered.

In accordance with a preferred embodiment, at least one ring element can have a gap at the periphery, such that the ends at the periphery are distanced. In particular, in accordance with a preferred embodiment, at least two ring elements can be arranged concentrically. This is particularly expedient in the case of ring elements that have a gap in the periphery. However, a concentric arrangement of ring elements with overlapping ends can be used in other embodiments.

In accordance with a preferred embodiment, the elements and/or the longitudinal element may also be metallic. The insertion element has good flexibility and also very good “trackability” in the body, that is to say the insertion element can be discovered as a result of the metallic constituents, even if it is not visible from the outside.

In accordance with a preferred embodiment, the sleeve is a polyamide elastomer and/or polyurethane and/or silicone. These are suitable materials for flexible ports.

In accordance with a preferred embodiment, the sleeve is composed in the radial direction of an inner part and an outer part, between which the frame is arranged. This allows a preferred embodiment of the sleeve, with which desirable properties in the inner region for the passage of the implant and of the catheter through the insertion element and in the outer region for the insertion of the insertion element into the body can be provided practically independently of one another.

In accordance with a preferred embodiment, the inner part is a low-friction polymer, in particular PTFE (polytetrafluoroethylene), in particular Teflon®.

In accordance with a preferred embodiment, the outer part is a thermoplastic polymer, in particular a polyether block amide.

In accordance with a preferred embodiment, a friction-reducing coating is provided. The coating can be provided on the inner diameter and/or on the outer diameter of the insertion element. This facilitates the handling of the insertion element under conditions of use.

In accordance with a further aspect of the invention, an insertion device for the insertion of a medical implant into a human and/or animal body is provided including at least one outer insertion element, wherein the outer insertion element has a sleeve having a longitudinal axis, wherein a frame having elements arranged transversely to the longitudinal axis, at least in regions, and following one another along the longitudinal axis is integrated into the sleeve, the frame having a higher hardness than the sleeve. Under conditions of use, the handling of the insertion device is facilitated.

In the figures, functionally like or similarly acting elements or components are denoted in each case by like reference signs. The figures are schematic illustrations of the invention. They do not show specific parameters of the invention. The figures also merely reproduce typical embodiments of the invention and are not intended to limit the invention to the embodiments illustrated.

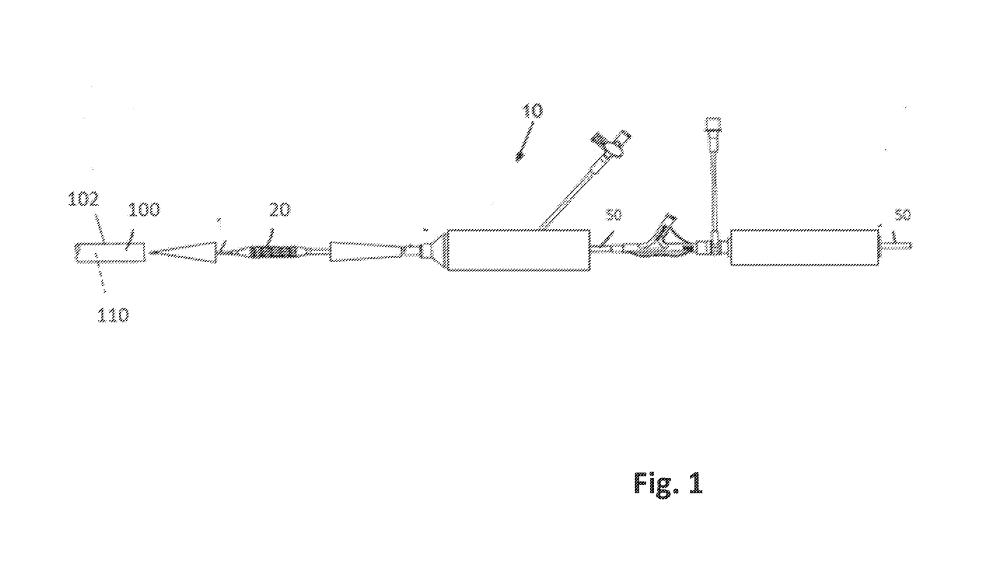

FIG. 1 shows a view of an insertion device 10 in accordance with an exemplary embodiment of the invention with an insertion element 100, through which an implant 20 fastened to a catheter 50 of the insertion device is introduced into a human and/or animal body. The insertion element 100 has a sleeve 102, in which a frame 110 is integrated, which has a higher hardness than the sleeve 102. The insertion element thus obtains an improved stability in the direction of its longitudinal axis and is more stable with respect to compression and tension, which is advantageous when introducing and removing the insertion device 10.

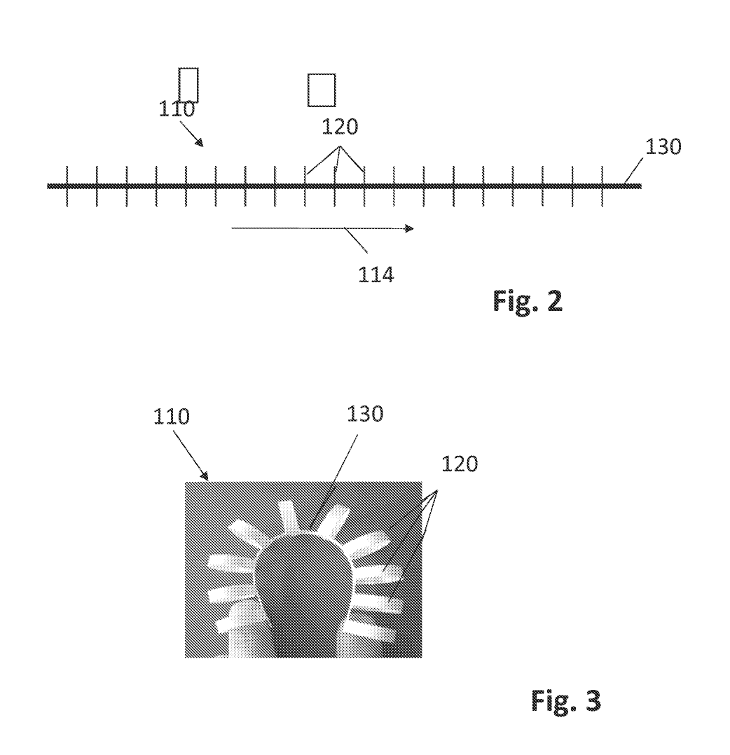

An example of a frame 110 is illustrated in FIG. 2 as a plan view. As can be seen in FIG. 2, the transverse elements 120 are arranged transversely to the longitudinal axis 114 and following one another along the longitudinal axis 114 and are connected to a longitudinal element 130 that extends in the direction of the longitudinal axis 114. Here, the longitudinal axis 114 is the longitudinal extension of the insertion element 100 in the rest state. In use, the insertion element 100 can be curved, for example in order to enable access for the insertion device 10 into a bodily vessel. The transverse elements 120 can be straight or may have other, curved cross sections as ring elements.

FIG. 3 shows the flexibility of the frame 110 when the longitudinal element 130 is curved. The longitudinal element 130 stabilizes the insertion element 100 against tension and compression, even in the curved state.

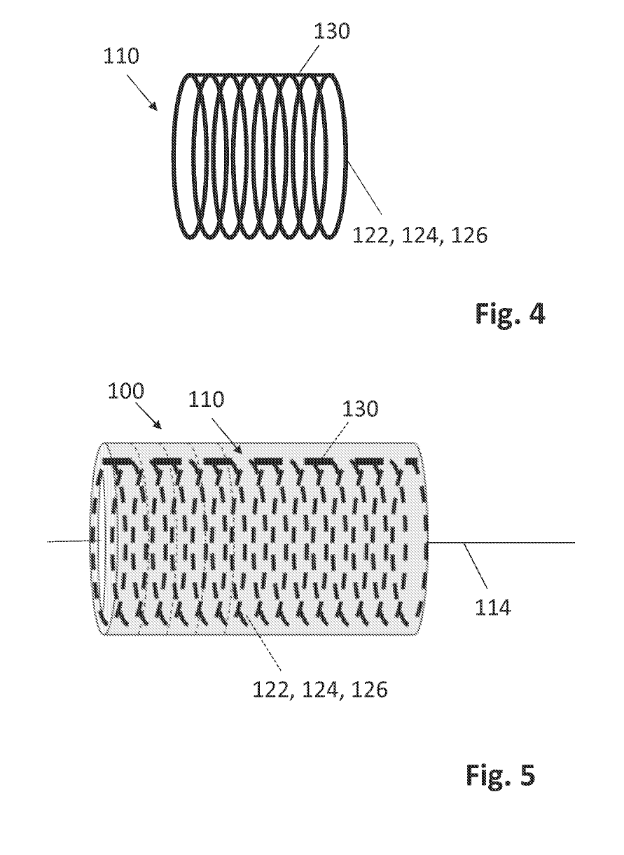

FIG. 4 shows, in side view, a frame 110 with transverse elements 120 formed as ring elements 122, 124, 126, which are connected by means of a longitudinal element 130. The ring elements 122, 124, 126 can preferably be expanded resiliently.

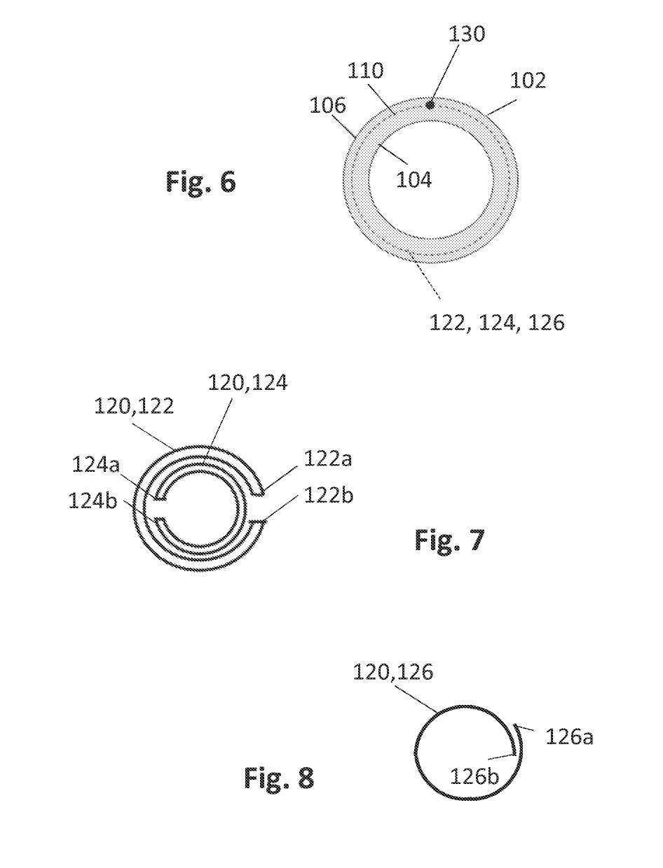

FIG. 5 shows, in side view, an insertion element 100 with a sleeve 102 and indicated frame 110 in the sleeve 102. FIG. 6 shows a plan view of a cross section through the insertion element 100 from FIG. 5. The longitudinal element 130 extends in the direction of the longitudinal axis 114. In the case of a longitudinal bending of the insertion element 100, the longitudinal axis 114 corresponds to the chord of the sleeve 102.

The sleeve 102 is composed in the radial direction of an inner part 104 and an outer part 106, between which the frame 110 is arranged. The inner part 104 comprises a low-friction polymer, in particular PTFE, such as Teflon, whereas the outer part 106 comprises a thermoplastic polymer, in particular a polyether block amide, such as Pebax® from the company Arkema. Alternatively or additionally, a friction-reducing coating with a hydrophilic or hydrophobic material can also be provided internally and/or externally (not illustrated).

FIGS. 7 and 8 show exemplary transverse elements 120, which are formed as resiliently expandable, open ring elements 122, 124, 126. An insertion element 100 formed as a port with a resilient radially expandable design can thus be presented.

FIG. 7, in plan view, shows a detail of a frame 110 in accordance with an embodiment of the invention, in which expandable ring elements 122, 124 are arranged concentrically with gap in the periphery. The ring elements 122, 124 are curved along an almost closed periphery, such that a gap is formed in the periphery in each case and the ends 122a, 122b and 124a, 124b are distanced from one another. The gaps in the periphery of a ring element 122, 124 are covered by the other ring element 124, 122.

Another variant is illustrated in FIG. 8, in which a ring element 126 has two ends 126a, 126b overlapping at its periphery.

Different ring elements 122, 124, 126 can also be combined with one another in a frame 110. A frame 110 in which straight and curved elements 120 are combined with one another is provided in another embodiment. The frame 110 is preferably formed from metal, for example from a spring steel.

The dimensions of the sleeve 102 and of the frame 110 can be set and varied during production. By way of example, values for a conventional port are specified: a ring element 122, 124, 126 made of steel can be provided with a diameter with 4 mm, which for example can be expanded to 6 mm. The radial thickness of the ring element 122, 124, 126 may be 0.02 mm, whereas the axial extension may be 3 mm, for example. The distance between axially adjacent ring elements 122, 124, 126 can be 3 mm. The longitudinal element can be formed from spring steel with a diameter of 0.01 mm, for example. The frame can be coated, for example with a Pebax® layer 0.03 mm thick. The sleeve 102 advantageously consists of a polyamide elastomer and/or polyurethane and/or silicone.

This invention allows a design for a tube-like insertion element with versatile flexibility and at the same time a high axial strength of the insertion element.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. An insertion element for cooperation with an insertion device for the insertion of a medical implant into a human or animal body, the insertion element comprising a sleeve having a longitudinal axis, a frame having, at least in regions, transverse elements arranged transversely to the longitudinal axis and following one another along the longitudinal axis, the frame being integrated into the sleeve, and a longitudinal element connecting said transverse elements along the longitudinal axis, wherein said frame has a higher hardness than the sleeve.

2. The insertion element as claimed in claim 1, wherein the longitudinal element is resilient and bendable.

3. The insertion element as claimed in claim 1, wherein at least one of the transverse elements is formed as a ring element, which can be resiliently expanded in the radial direction.

4. The insertion element as claimed in claim 3, wherein at least one of the ring elements is curved along a closed or almost closed periphery having two ends separated from one another.

5. The insertion element as claimed in claim 3, wherein at least one ring element has two ends overlapping at its periphery.

6. The insertion element as claimed in claim 3, wherein at least one ring element has a gap at the periphery, such that the ends are distanced at the periphery.

7. The insertion element as claimed in claim 3, comprising at least two ring elements are arranged concentrically.

8. The insertion element as claimed in claim 1, wherein the transverse elements and/or the longitudinal element are metallic.

9. The insertion element as claimed in claim 1, wherein sleeve comprises a polyamide elastomer and/or polyurethane and/or silicone.

10. The insertion element as claimed in claim 1, wherein the sleeve is composed in the radial direction of an inner part and an outer part, between which the frame is arranged.

11. The insertion element as claimed in claim 10, wherein the inner part comprises a low-friction polymer.

12. The insertion element as claimed in claim 11, wherein the inner part comprises polytetrafluoroethylene.

13. The insertion element as claimed in claim 12, wherein the outer part comprises a thermoplastic polymer.

14. The insertion element as claimed in claim 13, wherein the outer part comprises a polyether block amide.

15. The insertion element as claimed in claim 11, wherein the outer part comprises a thermoplastic polymer.

16. The insertion element as claimed in claim 15, wherein the outer part comprises a polyether block amide.

17. The insertion element as claimed in claim 1, further comprising a friction-reducing coating.

18. An insertion device for the insertion of a medical implant into a human and/or animal body, comprising at least one outer insertion element in accordance with claim 1.
